(19) United States
(12) Patent Application Publication
(10) Pub. No.: US 2011/0060267 A1
(43) Pub. Date: Mar. 10, 2011

(54) ULTRAVIOLET IRRADIATION TO TREAT CORNEAL WEAKNESS DISORDERS

(76) Inventors: Bruce DeWoolfson, Vienna, VA (US); Dale DeVore, Chelmsford, MA (US)

(21) Appl. No.: 12/922,424

(22) PCT Filed: Mar. 10, 2009
(86) PCT No.: PCT/US09/36636

§ 371 Date: Nov. 22, 2010

Related U.S. Application Data
(60) Provisional application No. 61/064,600, filed on Mar. 14, 2008.
(60) Provisional application No. 61/064,864, filed on Mar. 31, 2008.
(60) Provisional application No. 61/071,580, filed on May 7, 2008.

(51) Int. Cl. A61F 9/00

(52) U.S. Cl. 604/20

(57) ABSTRACT

Methods of strengthening the biomechanical properties of the cornea by exposing the cornea to ultraviolet light in the presence of a photoinitiator are described. These methods can be used to treat keratoconus. They can also be used to treat ectasia following a surgical procedure, or to strengthen the cornea prior to a surgical procedure.

This application claims priority to U.S. Provisional Application Nos. 61/064,600 filed Mar. 14, 2008, 61/064,864 filed Mar. 31, 2008, and 61/071,580 filed May 7, 2008, the contents of which are all incorporated herein by reference.

### FIELD OF INVENTION

The present disclosure relates to a process for selectively treating cornea to strengthen the biomechanical properties of the tissue. More particularly, the disclosure provides a process for selectively treating in vivo animal tissue by exposing the cornea to ultraviolet irradiation in the presence of a photoinitiator to crosslink collagen and stabilize said tissue. This process may be used to increase the strength of cornea. Such treatment will provide therapeutic treatment of cornea weakness disorders including keratoconus and keratectasia. In addition, ultraviolet irradiation of corneal collagen will stabilize reshaped cornea following orthokeratology lens wear to provide long-term correction of myopia and other vision errors.

### BACKGROUND OF THE INVENTION

Keratoconus is characterized by generalized thinning and cone-shaped protrusion of the central cornea, which affects visual acuity. In the last stage, most cases need keratoplasty with all the risks associated with this procedure. Normally, this corneal disease affects both eyes but in different dimensions and at different times. Symptoms of keratoconus are: changing visual acuity despite correction with glasses or contact lenses, perception of halos around light sources, as well as increased sensitivity to light and blinding.

Keratoconus affects one in 2,000 people. Causes for this disease are unknown. In families which are affected, it occurs more often, so the reason might be genetic predisposition. Also, frequent and intense rubbing of the eyes for years, e.g., because of allergic reaction, is discussed as one possible reason for the development of keratoconus.

Progressive keratoconus is aggressive and can begin at a very early age. With progression of the disease, correction of visual acuity with glasses becomes more difficult because protrusion of the cornea develops unevenly. Hard contact lenses are a good solution because they put pressure on the cornea, thus correcting irregularities. If protrusion of the cornea continues, there will come a point when the patient cannot wear contact lenses any longer and the cornea becomes continuously thinner. In the region of ectasia, it can break through and develop scars. Visual acuity will be permanently worse. At the moment, there is no therapy that is successful in stopping or slowing the progression of the disease. Keratoconus cannot be healed. The only successful long-term treatment is keratoplasty, which means surgery with all included risks and complications. The patient regains an acceptable visual acuity often only months after surgery.

Corneal ectasia has been identified as a potential side effect of corneal refractive surgery. The incidence of post-surgical ectasia ranges from 1 in 2500 to 6 in 1000 patients. Ectasia is specifically associated with LASIK because LASIK penetrates the cornea much more deeply than other procedures (due to the thick stromal flap) and therefore can result in excessive thinning and structural compromise of the cornea. Ectasia is caused by biomechanical weakening or destabilization of the cornea due to excessive removal of tissue and disruption to the structure of the cornea.

Treatments for progressive keratoconus include penetrating keratoplasty, implantation of corneal rings (Intacs), and more recently, exposure to ultraviolet (UV) irradiation in combination with riboflavin. The latter treatment requires debridement of epithelium and long-term exposure to ultraviolet light. These same treatments have been used to treat corneal ectasia.

The present disclosure describes methods for treating progressive keratoconus and corneal ectasia using short-term exposure to ultraviolet light in combination with a simple photoinitiator. The same technique is also used to stabilize corneal structure prior to corneal surgery and following orthokeratology.

### Crosslinking Using Ultraviolet Irradiation

It is known that UV radiation and UVC is effective in crosslinking collagen. Kelman and DeVore have a number of patents describing the application of ultraviolet irradiation to crosslink or polymerize collagenous constructs. These patents, and additional patents of interest, are disclosed below.

U.S. Pat. No. 4,969,912 describes the application of ultraviolet (UV) to crosslink a collagen mass injected into the lamellae of the cornea resulting in a reshaped anterior curvature.

U.S. Pat. No. 5,067,961 describes the fabrication of a non-biodegradable corneal implant by exposing collagenous compositions to ultraviolet irradiation.

U.S. Pat. No. 5,104,957 and U.S. Pat. No. 5,480,427 describe the fabrication of formed medical implants and transplant articles by exposing molded and dehydrated collagen-based compositions to ultraviolet irradiation.

U.S. Pat. Nos. 5,219,895 and 5,874,537 describe collagen-based compositions that when exposed to ultraviolet irradiation (curing) form effective tissue sealants and adhesives. Curing was achieved by exposing compositions to short wave length irradiation in the presence of a photo-initiator such as sodium persulfate, sodium thiosulfate, ferrous chloride tetrahydrate, sodium bisulfite and oxidative enzymes such as peroxidase or catechol oxidase. When initiators were employed, polymerization or curing occurred in 30 seconds to 5 minutes, usually from 1 to 3 minutes.

DeVore, Putnam, and Pachence (U.S. Pat. No. 6,183,498) described methods and products for sealing a fluid leak in a tissue by exposing chemically modified collagen solution to polymerization or crosslinking conditions to produce the polymerized collagen composition. Polymerization was carried out using irradiation, e.g., UV, gamma, or fluorescent light. In one embodiment, the polymerizable protein was in a solvent which includes an initiator. The initiator can be sodium persulfate, sodium thiosulfate, ferrous chloride tetrahydrate, sodium bisulfate or an oxidative enzyme.

DeVore and Oefinger (U.S. Pat. No. 6,161,544) described the polymerization or crosslinking of reshaped corneal tissues by exposing the cornea to short wave UV light (e.g. 254 nm). However, it was found that the rate of polymerization was not practical for use because of the potential damage to the corneal tissues caused by long term exposure to UV light. The rate of polymerization was significantly increased by applying appropriate redox initiators to the cornea prior to the UV light exposure. Suitable, but non-limiting, examples of some initiators include sodium persulfate, sodium thiosulfate, ferrous chloride tetrahydrate, sodium bisulfate, and oxidative enzymes such as peroxidase or catechol oxidase.

El Hage (WO 2007/082127 A2) describes methods to provide long lasting and potentially permanent reshaping the curvature of the cornea using a combination of “controlled kerato-reformation” or orthokeratology with riboflavin and ultraviolet light. The method includes debridement of epithelium and exposure times of at least 30 minutes.

Hamed and Rodriguez, J. Applied polymer Sci., 19:3299-3313, 1975 reported gelation of telopeptide-poor collagen solutions exposed to 254 nm ultraviolet irradiation in a nitrogen atmosphere.

Weadock, et. al., J. Biomed. Mat. Res. 29: 1373-1379, 1995 reported that the ultimate tensile strength and modulus values of collagen fibers extruded from an acid solution and then exposed to 254 nm ultraviolet irradiation were slightly greater or equivalent to collagen fibers crosslinked using dehydrothermal methods.

Weadock, et. al., J. Biomed. Mat. Res. 32: 221-226, 1996 reported that insoluble collagen fibers extruded from an acid solution exhibited increased shrinkage temperature, resistance to collagenase and durability under load in collagenase following 254 nm ultraviolet irradiation.

Lew, et. al., J Biomed Mater Res B Appl Biomater. July; 82(1):51-6, 2007 reported that artificial collagen-based matrices exposed to ultraviolet irradiation or a combination of ultraviolet irradiation and dehydrothermal crosslinking exhibited physical durability and cell compatibility.

Ohan, et. al., J Biomed Mater Res. Jun. 5; 60(3):384-91, 2002 reported that collagen films exposed to ultraviolet irradiation with glucose exhibited improved mechanical properties, enzyme resistance without significant denaturation effects.

DeVore (Encyclopedic Handbook of Biomaterials and Bioengineering, Eds. Wise, et. al., Marcel Dekker, New York, 1995) and Kornmehl, et. al. (Refract. Surg. 11: 502-506, 1995) described methods of preparing artificial corneal grafts by exposing collagen-based dehydrated films to 254 nm ultraviolet irradiation. Grafts were stable in rabbit eyes for more the 1-month evaluation period.

Each of the listed patents and publications, with the exception of DeVore and Oefinger (U.S. Pat. No. 6,161,544) describe the application of ultraviolet irradiation to crosslink or polymerize collagen or collagen-based constructs. DeVore and Oefinger also describe the application of ultraviolet irradiation, in the presence of a photo-initiator, to stabilize animal cornea following remolding. However, the patent does not discuss or describe the application of ultraviolet irradiation to strengthen weakened cornea resulting from keratoconus or post-surgical ectasia.

### Crosslinking Using Riboflavin and Ultraviolet Irradiation

There are a number of publications reporting the application of ultraviolet light with the photoinitiator riboflavin for treatment of keratoconus and for stopping the progression of ectasia following LASIK procedures.

Wollensak, et. al., Am J Ophthalmol. 135(5):620-7, 2003 and Wollensak, et. al., Ophthalmologe. 100(1):44-9 2003 reported a significant increase in corneal biomechanical stiffness after collagen crosslinking by combined riboflavin/ultraviolet-A (UVA) treatment. Treated cornea were ablated, and exposed to UVA for 30 minutes after applying riboflavin drops.

Kanellopoulos and Binder, Cornea, 26(7):891-5, 2007 reported successful clinical treatment of keratoconus using UVA irradiation (3 mW/cm for 30 minutes) after topical 0.1% riboflavin drops over a deepithelialized cornea.

The application of ultraviolet irradiation in combination with riboflavin appears to successfully treat keratoconus by increasing the corneal biomechanical stiffness. However, the treatment requires removal of epithelium and takes 30 minutes to accomplish effective crosslinking.

### SUMMARY OF THE INVENTION

The disclosure describes methods to treat weakened or thinned cornea by exposing the cornea to ultraviolet irradiation in the presence of a photoinitiator, such as sodium persulfate. It also provides methods to strengthen the cornea prior to corneal surgery. The method does not require removal of epithelium. In one embodiment, the method takes less than 1 minute of exposure time to ultraviolet irradiation.

### BRIEF DESCRIPTION OF THE DRAWINGS

FIG. 1 shows the effects of multiple ultraviolet irradiation exposures on the low modulus of porcine cornea.

### DEFINITIONS

“Stabilization” refers to the increase in mechanical properties of treated cornea.

“Crosslinking” or “polymerization” refers to the formation of chemical links between the molecular chains in polymers, such as collagen fibers.

“Photoinitiator” refers to an agent which when exposed to a specific wavelength of energy forms a reactive element which starts the chain reaction to cause polymerization of molecular chains in polymers. Examples include sodium persulfate, sodium thiosulfate, ferrous chloride tetrahydrate, sodium bisulfate.

### DETAILED DESCRIPTION

The present disclosure provides methods for treating intact cornea with ultraviolet irradiation in the presence of a photoinitiator to increase the mechanical properties of weakened cornea. Such treatment will treat keratoconus and ectasia, and can also be used to strengthen the cornea prior to corneal surgery.

Two major conditions resulting in weakened cornea include keratoconus and ectasia. Keratoconus is a degenerative disease of the cornea that causes it to gradually thin and bulge into a cone-like shape. This shape prevents light from focusing precisely on the macula. As the disease progresses, the cone becomes more pronounced, causing vision to become blurred and distorted. Because of the cornea's irregular shape, patients with keratoconus are usually very nearsighted and have a high degree of astigmatism that is not correctable with glasses.

Ectasia or keratoectasia is a bulging of the corneal. Ectasia is also called iatrogenic keratoconus or secondary keratoconus because it is basically a surgically induced version of the naturally occurring disease keratoconus. Ectasia is a very serious long-term complication of LASIK. Ectasia is specifically associated with LASIK because LASIK penetrates the cornea much more deeply than other procedures (due to the thick stromal flap) and therefore can result in excessive thinning and structural compromise of the cornea. Ectasia is caused by biomechanical weakening or destabilization of the cornea due to excessive removal of tissue and disruption to the structure of the cornea.

Ectasia following corneal surgery may be prevented, or at least reduced, by strengthening the cornea with ultraviolet irradiation in the presence of a photoinitiator prior to the surgery.

The inventors have discovered that the application of ultraviolet irradiation in the presence of a simple photo-initiator such as sodium persulfate can significantly increase the mechanical strength of exposed cornea.

Thus, in one embodiment, the disclosure provides a method of treating keratoconus, comprising applying a photoinitiator to the keratoconic cornea and exposing the cornea to ultraviolet irradiation for a period of equal to or less than about 10 minutes. In other embodiments, the disclosure provides methods of treating ectasia following a corneal surgery, comprising applying a photoinitiator to the ectasic cornea and exposing the cornea to ultraviolet irradiation for a period of equal to or less than about 10 minutes. In still other embodiments, the disclosure provides methods of strengthening a cornea prior to a corneal surgery, comprising applying a photoinitiator to the cornea and exposing the cornea to ultraviolet irradiation for a period of equal to or less than about 10 minutes, followed by the corneal surgery.

Exposure of the cornea to the ultraviolet irradiation can be for an uninterrupted period of time, or it can occur in bursts of shorter exposures times. Individual exposure times can be about 1, 2, 3, 4, 5, 10, 15, 20, 25, 30, 35, 40, 45, 50, or 55 seconds in length, or even for about 1, about 2, about 3, about 4, about 5, about 6, about 7, about 8, about 9, or about 10 minutes. The duration may also fall within a range set by any two of the aforementioned values.

The photoinitiators used in the methods of the invention generally, but not always, are water soluble, activated by UV, and have limited or no toxicity. Some examples of photoinitiators include, but are not limited to, sodium persulfate, potassium persulfate, ammonium persulfate, sodium thiosulfate, ferrous chloride tetrahydrate, or sodium bisulfate. In some embodiments, the photoinitiator is sodium persulfate. In certain embodiments, the photoinitiator is not riboflavin.

Selection of an appropriate photoinitiator depends on the water solubility, irradiation wavelength, and biocompatibility of the photoinitiator at the concentration required for effective polymerization.

In certain embodiments, the UV photoinitiators are water soluble. Examples of water soluble initiators include ammonium persulfate, potassium persulfate, sodium persulfate, sodium thiosulfate and the like, and redox-type initiators which are combinations of such initiator and tetramethylethylene, sodium hydrogen sulfite or like reducing agent, etc.

Photoinitiators include the photoinitiating dyes. Photoinitiating dyes capture light energy and initiate polymerization of proteins and other macromolecular entities. Suitable UV wavelengths range from about 200 to about 400 nm. Any dye can be used which absorbs light having frequency between about 200 nm and 700 nm, can form free radicals, is at least partially water soluble, and is non-toxic to the biological material at the concentration used for polymerization. Examples of suitable dyes include but are not limited to ethyl eosin, eosin Y, fluorescein, 2,2-dimethoxy, 2-phenylacetophenone, 2-methoxy, 2-phenylacetophenono, camphorquinone, rose bengal, methylene blue, erythrosin, phloxime, thionine, riboflavin, and methylene green. In certain embodiments, the dye is not riboflavin.

Additional initiators include compounds such as lauryl peroxide, benzoyl peroxide, isopropyl percarbonate, azobisisobutyronitrile, and the like, that generate free radicals at moderately elevated temperatures, and photoinitiator systems such as aromatic alpha-hydroxy ketones, alkoxyoxybenzoins, acetophenones, and acyl phosphine oxides, and the like. Some specific examples of these types of photoinitiators are 1-hydroxycyclohexyl phenyl ketone, 2-hydroxy-2-methyl-1-phenyl-propan-1-one, bis(2,6-dimethoxybenzoyl)-2,4-4-trimethylpentyl phosphine oxide (DMBAPO), bis(2,4,6-trimethylbenzoyl)-phenylphosphineoxide (Irgacure 819), 2,4,6-trimethylbenzyldiphenyl phosphine oxide and 2,4,6-trimethylbenzyoyl diphenylphosphine oxide, benzoin methyl ester, and a combination of camphorquinone and ethyl 4-(N,N-dimethylamino)benzoate.

Still other UV photoinitiators include 2,2-dimethoxy-2-phenyl acetophenone, benzoin ethyl ether, 2,2-dimethyl phenoxyacetophenone, benzophenones, benzils, and thioxanthones. In some embodiments, ionic derivatives of the photoinitiators are to improve their water solubility.

Commercially available UV photoinitiators can also be used. Examples include Darocur 1173 and Darocur/Ingracure 2959 (Ciba Specialty Chemicals). The initiator is used in effective amounts to initiate photopolymerization of the reaction mixture. Polymerization of the reaction mixture can be initiated using the appropriate choice of heat or visible or ultraviolet light or other means depending on the polymerization initiator used.

A list of photoinitiators available from Sigma-Aldrich is shown in Table 1. One or more of the compounds in Table 1 can be in the disclosed methods, either alone, together, or in combination with one or more of the other photoinitiators described.

TABLE 1
Sigma Aldrich Initiators
A PhotoInitiators

| Product # | Product Name |
|---|---|
| A10701 | Acetophenone ReagentPlus, 99% |
| 415952 | Diphenyl(2,4,6-trimethylbenzoyl)phosphine oxide 97% |
| A88409 | 4,4′-Dimethoxybenzoin 95% |
| A90004 | Anthraquinone 97% |
| 123242 | Anthraquinone-2-sulfonic acid Sodium salt 97% |
| 119318 | Benzene-chromium(0) tricarbonyl 98% |
| B5151 | 4-(Boc-aminomethyl)phenyl isothiocyanate ~95% |
| B5151 | Benzil 98% |
| 399396 | Benzoin purified by sublimation, ≧99.5% |
| 172006 | Benzoin ethyl ether 99% |
| 195782 | Benzoin isobutyl ether technical grade, 90% |
| B8703 | Benzoin methyl ether 96% |
| B9300 | Benzophenone ReagentPlus, 99% |
| B9300 | Benzoic acid meets USP testing specifications |
| 405620 | Benzophenone/1-hydroxycyclohexyl phenyl ketone, 50/50 blend |
| 262463 | Benzophenone-3,3′,4,4′-tetracarboxylic dianhydride 98%, purified by sublimation |
| B12601 | 4-Benzoylbiphenyl 99% |
| 405647 | 2-Benzyl-2-(dimethylamino)-4′-morpholinobutyrophenone 97% |
| 160326 | 4,4′-Bis(diethylamino)benzophenone ≧99% |
| 147834 | Michler's ketone 98% |
| 124893 | (±)-Camphorquinone 97% |
| C72404 | 2-Chlorothioxanthen-9-one 98% |
| D31737 | 5-Dibenzosuberenone 97% |
| 227102 | 2,2-Diethoxyacetophenone >95% |
| D110507 | 4,4′-Dihydroxybenzophenone 99% |
| 196118 | 2,2-Dimethoxy-2-phenylacetophenone 99% |
| 149349 | 4-(Dimethylamino)benzophenone 98% |
| 146706 | 4,4′-Dimethylbenzil 97% |
| D149675 | 3,4-Dimethylbenzophenone 99% |
| 405663 | Diphenyl(2,4,6-trimethylbenzoyl)phosphine oxide/2-hydroxy-2-methylpropiophenone, blend |
| 275719 | 4′-Ethoxyacetophenone 98% |
| E12206 | 2-Ethylanthraquinone ≧97% |
| F408 | Ferrocene 98% |
| 328103 | 3′-Hydroxyacetophenone ≧99% |
| 278564 | 4′-Hydroxyacetophenone 99% |
| 220434 | 3-Hydroxybenzophenone 99% |
| H20202 | 4-Hydroxybenzophenone 98% |
| 405612 | 1-Hydroxycyclohexyl phenyl ketone 99% |
| 405655 | 2-Hydroxy-2-methylpropiophenone 97% |
| 157538 | 2-Methylbenzophenone 98% |
| 198056 | 3-Methylbenzophenone 99% |
| M30507 | Methyl benzoylformate 98% |
| 405639 | 2-Methyl-4′-(methylthio)-2-morpholinopropiophenone 98% |
| 156507 | 9,10-Phenanthrenequinone ≧99% |
| 290742 | 4′-Phenoxyacetophenone 98% |
| T34002 | Thioxanthen-9-one 97% |
| 407216 | Triarylsulfonium hexafluorophosphate salts, mixed 50% in propylene carbonate |
| 405736 | 3-Mercapto-1-propanol 95% |
| 447528 | 11-Mercapto-1-undecanol 97% |
| 328375 | 1-Mercapto-2-propanol 95% |
| 264792 | 3-Mercapto-2-butanol, mixture of isomers 97% |

B. Thermal Initiators

| | | | | | 10th Half-life |
|---|---|---|---|---|---|
| Product # | Product Name | Solvent | T (° C.) | k_{d} (s⁻¹) | ° C. (Solvent) |
| 118168 | 4,4′-Azobis(4-cyanovaleric acid) ≧75% | Acetone | 70 | 4.6 × 10⁻⁵ | 69 (water) |
| | | Water | 69 | 1.9 × 10⁻⁵ | |
| | | Water | 80 | 9.0 × 10⁻⁵ | |
| 380210 | 1,1′-Azobis(cyclohexanecarbonitrile) 98% | Toluene | 80 | 6.5 × 10⁻⁵ | 88 (toluene) |
| | | | 95 | 5.4 × 10⁻⁵ | |
| | | | 102 | 1.3 × 10⁻⁴ | |
| 441090 | 2,2′-Azobis(2-methylpropionitrile) 98% | Benzene | 50 | 2.2 × 10⁻⁶ | 65 (toluene) |
| | | | 70 | 3.2 × 10⁻⁵ | |
| | | | 100 | 1.5 × 10⁻³ | |
| 179981 | Benzoyl peroxide reagent grade, 97% | Benzene | 60 | 2.0 × 10⁻⁶ | 70 (benzene) |
| | | | 78 | 2.3 × 10⁻⁵ | |
| | | | 100 | 5.0 × 10⁻⁴ | |
| 441694 | 2,2-Bis(tert-butylperoxy)butane Solution 50 wt. % | | | | 100 (benzene) |
| | in mineral oil | | | | |
| 388092 | 2,5-Bis(tert-butylperoxy)-2,5-dimethylhexane | Benzene | 93 | 1.9 × 10⁻⁵ | 120 (benzene) |
| | technical grade, 90% | | | | |
| 441716 | Bis[1-(tert-butylperoxy)-1-methylethyl]benzene | | 115 | 1.1 × 10⁻⁵ | 115 (benzene) |
| | 96% | | 145 | 4.7 × 10⁻⁴ | |
| 416665 | tert-Butyl hydroperoxide Solution 5.0-6.0M in | Benzene | 130 | 3 × 10⁻⁷ | 170 (benzene) |
| | decane | | 160 | 6.6 × 10⁻⁶ | |
| | | | 170 | 2.0 × 10⁻⁵ | |
| | | | 183 | 3.1 × 10⁻⁵ | |
| 388076 | tert-Butyl peracetate Solution 50 wt. % in odorless | Benzene | 85 | 1.2 × 10⁻⁶ | 100 (benzene) |
| | mineral spirits | | 100 | 1.5 × 10⁻⁵ | |
| | | | 130 | 5.7 × 10⁻⁴ | |
| 168521 | tert-Butyl peroxide 98% | Benzene | 80 | 7.8 × 10⁻⁸ | 125 (benzene) |
| | | | 100 | 8.8 × 10⁻⁷ | |
| | | | 130 | 3.0 × 10⁻⁵ | |
| 159042 | tert-Butyl peroxybenzoate 98% | Benzene | 100 | 1.1 × 10⁻⁵ | 103 (benzene) |
| | | | 130 | 3.5 × 10⁻⁴ | |
| 247502 | Cumene hydroperoxide technical grade, 80% | Benzene | 115 | 4.0 × 10⁻⁵ | 135 (toluene) |
| | | | 145 | 6.6 × 10⁻⁴ | |
| 329541 | Dicumyl peroxide 98% | Benzene | | | 115 (benzene) |
| 290785 | Lauroyl peroxide 97% | Benzene | 40 | 4.9 × 10⁻⁸ | 65 (benzene) |
| | | | 60 | 9.2 × 10⁻⁷ | |
| | | | 85 | 3.8 × 10⁻⁵ | |
| 269336 | Peracetic acid Solution 32 wt. % in dilute acetic | | | | 135 (toluene) |
| | acid | | | | |
| 216224 | Potassium persulfate ACS reagent, ≧99.0% | Water | 80 | 6.9 × 10⁻⁸ | 60 (H₂O) |
| | | 0.1M | 50 | 9.5 × 10⁻⁷ | 70 (0.1M NaOH) |
| | | NaOH | 60 | 3.2 × 10⁻⁶ | |
| | | | 80 | 9.2 × 10⁻⁵ | |
| | | | 90 | 3.5 × 10⁻⁴ | |

The ultraviolet irradiation used in the methods generally has a wave length in the range of about 200 nm to about 400 nm. The wavelength or wave length range chosen will depend in part on the distance of the UV source from the patient's eye. Thus, wave lengths of about 200 nm, about 250 nm, about 300 nm, about 350 nm, about 400 nm, or a wave length range that falls between any of these values can be used, depending upon the embodiment. In certain embodiments, the ultraviolet irradiation has a wave length of about 254 nm to about 400 nm. In some embodiments, a wave length of about 254 nm is used. In still other embodiments, the ultraviolet irradiation has a wave length of about 310 nm to about 400 nm.

The intensity of the ultraviolet light can vary, but generally it is in the range of about 100 mW to about 2000 mW. In certain embodiments, the intensity of the ultraviolet light is about 100 mW, 200 mW, about 300 mW, about 400 mW, about 500 mW, about 600 mW, about 700 mW, about 800 mW, about 900 mW, 1000 mW, or about 2000 mW, or the intensity may fall within a range that is set by any two of the aforementioned values between 100 and 2000. In certain embodiments, the intensity is about 1000 mW.

In some embodiments, the period of exposure comprises from 1 to 4 bursts of ultraviolet light exposure, wherein each burst of ultraviolet exposure is about 10 seconds in duration and the period of non-ultraviolet exposure between each burst is about 10 seconds. In certain embodiments, the period of ultraviolet light exposure consists of a single exposure of about 10 seconds. Other durations for the burst of ultraviolet light exposure are possible, such as 1, 2, 3, 4, 5, 10, 15, 20, 25, 30, 35, 40, 45, 50, or 55 second in length, or even for about 1, about 2, about 3, about 4, about 5, about 6, about 7, about 8, about 9, or about 10 minutes. The duration may also fall within a range set by any two of the aforementioned values.

In those embodiments involving methods of treating ectasia following a surgery or involving methods of strengthening the cornea prior to a surgery, the surgery can include Laser Assisted In Situ Keratomielusis (“LASIK”), Laser Epithelium Keratomileusis (“E-LASIK”), Conductive Keratoplasty (“Radiofrequency energy”), or Microwave Thermokeratoplasty.

Methods of treating ectasia generally are initiated after a diagnosis of ectasis is made by a qualified professional. Once ectasia has been diagnosed, the patient can then schedule to have the cornea treated with ultraviolet irradiation in the presence of a photoinitiator at a time of his or her choosing.

The methods of strengthening the cornea by cornea by treating it with ultraviolet irradiation in the presence of a photoinitiator prior to a surgery can be initiated at any of a variety of time points after the patient has been informed that surgery is needed, or informed that surgery is an option for that patient. For example, a patient considering LASIK may receive the strengthening treatment at the time of his or her LASIK prescreening examination. Alternatively, the strengthening treatment may be administered at a time between the prescreening exam and the surgery. In general, the strengthening treatment will take place within the month preceding the surgery, but of course in some cases the time period may be more than a month before the surgery. For example, it is possible that the strengthening treatment could be administered 5, 6, 7, 8, or even more weeks before. Usually, however, the strengthening treatment will be administered about one to two weeks before the corneal surgery. Often, the strengthening treatment will be administered about 10 days before the surgery, although it may be administered about 9, about 8, about 7, about 6, about 5, about 4, about 3, about 2, about 1 days before the corneal surgery. It is even possible to treat the cornea with the ultraviolet light in the presence of a photoinitiator on the same day as the corneal surgery.

The disclosure describes the controlled application of a pretreatment photoinitiator solution to the corneal surface followed by exposure to short wave length ultraviolet irradiation for as little as 10 seconds. The photoinitiator is administered by adding the solution to an applicator placed on the anterior corneal surface. The applicator controls the exposure area of the solution. Non-limiting examples of applicators for use in applying solutions to the corneal surface are described in the co-pending provisional application entitled “APPARATUS TO IMPROVE LOCALIZED CONCENTRATION OF FLUIDS IN OCULAR ENVIRONMENTS” to Bruce DeWoolfson and Michael Luttrell, provisional application No. 61/064,731, which is incorporated herein by reference in its entirety. Following exposure for about 30 seconds to about 1 minute, the corneal surface is exposed to ultraviolet irradiation administered via a light guide fitted to an applicator.

The concentration of the photoinitiator ranges from about 0.01M to 0.5M mixed in 0.05 to 0.5M sodium phosphate buffer. Often the concentration of the photoinitiator ranges from 0.025M to 0.3M and usually from 0.05M to 0.1M. In general, the concentration of the phosphate buffer ranges from about 0.1M to 0.4M and usually it is from 0.15M to 0.3M. The pH of the final solution generally ranges from about 6.5 to about 8.5. Often the pH is between about 7.0 to about 8.0 and usually it is between about 7.2 to about 7.8.

Polymerization or crosslinking by ultraviolet irradiation may be accomplished in the short wave length range using a variety of sources. For example, effective polymerizing of collagen films has been accomplished using a standard 254 nm source providing as little as 100.2 mW/cm² at the photodiode tube and 50 mW/cm² at the tissue surface. However, at this exposure level, it may take several minutes to effectively crosslink or polymerize corneal collagen.

Although not required, it is generally preferable to use a more efficient ultraviolet irradiation source such as an instrument providing 20,000 mW/cm2 of curing power (e.g., EFOS Novacure, Model N2000; EFOS Mississauga, Ontario Canada L5N 6H7). The intensity can be adjusted to lower levels as appropriate. A light intensity of 1000 mW will be described in the non-limiting Examples that follow. This intensity provided rapid polymerization of corneal structures. Because excess UV exposure will begin to depolymerize the collagen polymers and cause eye damage, it is important to limit UV irradiation for short periods. In the experiments outlined below, the UV exposure was conducted with no filter, thereby providing broadband UV irradiation. Filters will provide a more specific wavelength, which will be matched to an appropriate photochemical or redox initiator. Filters also reduce the temperature elevation at the exposure site. Sodium persulfate which is listed as the preferred initiator in Example 1 exhibits a maximum absorption at 254 nm, but appears to be effective at much higher wavelengths. For maximum efficiency, the UV wavelength should be matched to the specific initiator.

### EXAMPLES

### Example 1

Seven enucleated porcine eyes were placed in holders and the corneas flooded with 1.0 mL of 0.02M disodium phosphate (pH 8.5). The solution was removed using a surgical sponge and the corneas treated with three 1-minute exposures to acetic anhydride at 3 mg/mL (3 μL added to 1. mL of 0.02M disodium phosphate solution) to “soften” the tissue. The corneas were then flushed with neutral pH sodium phosphate buffer, at pH 7.2, for 1 minute and then treated with 0.35M sodium persulfate in phosphate buffer, pH 7.6-8.0 for 1 minute. Solutions were applied using a specially designed “staging device” to prevent leakage outside the surface of the cornea. Pretreated cornea were exposed to either one, three, and four 10-second bursts of UV light at an intensity of 1000 mW at a band pattern of 310-400 nm, with 10 second non-exposure bursts between UV exposure. After exposure, eyes were flushed with neutral pH phosphate buffer.

After treatment, corneal buttons were dissected, placed in Optisol and tested by stress-strain analysis. For stress-strain analysis, corneal buttons were placed on a slightly convex surface and exposed to compressive forces. Stress-strain curves represent the force/unit areas of cross-section required to compress the cornea a certain amount (%). Resultant curves consist of several distinct phases, the lower part (low modulus) representing the resistance to squeeze out fluid between collagen fibrils, a middle part wherein the stress-strain curve does not change and the upper part (high modulus region) representing compression of collagen fibrils. A reduction in low modulus indicates that the cornea is softer. An increase indicates that the corneal buttons are stiffer.

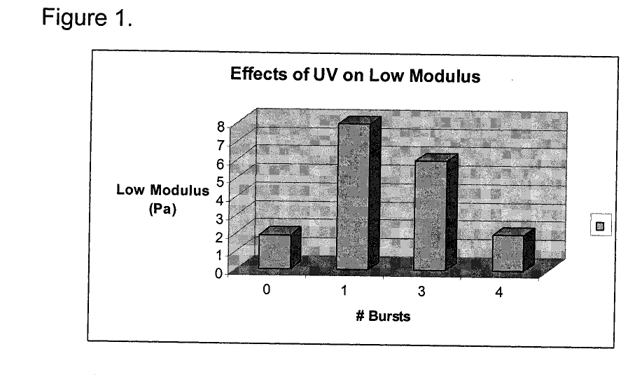

Stress-strain analysis showed that the low modulus increased following exposure to ultraviolet irradiation, indicating stiffening or strengthening of corneal buttons. As shown in FIG. 1 strengthening appeared to be maximum after a single exposure. Subsequent exposures reduced the increase in low modulus. After four exposures, the low modulus was nearly equal to untreated control cornea.

### Example 2

### Treatment of Keratoconus

A subject is diagnosed with keratoconus in both eyes. He is not able to wear contact lenses. Pachymetry of the cornea shows 320 microns at the weakest point. Corneal hysteresis measurements are made using the Reichert Ocular Response Analyzer. Corneal hysteresis (CH) is at least 3 numbers less than the normal population. Corneal topography is also conducted to identify the location of keratoconus. The subject is chosen to receive treatment using short wavelength ultraviolet irradiation following administration of a low concentration photoinitiator. Drops of 0.35M sodium persulfate in 0.02M sodium phosphate buffer at a pH of 7.6-8.0 are administered to the cornea in an applicator designed to limit exposure to the location of keratoconus on the corneal surface. The cornea is then exposed to one 10-second burst of ultraviolet irradiation at an intensity of 1000 mW.

After treatment, the subject shows significant improvement in vision and an increase in CH, indicating an increase in the biomechanical integrity of the cornea.

### Example 3

### Treatment for Post-LASIK Ectasia

A subject is diagnosed with ectasia following a LASIK procedure. Examinations show thinning and progressive central and inferior steepening of the cornea. Mechanical stability is measured using the Reichert Ocular Response Analyzer. CH values correlate with reduced mechanical stability. Pachymetry of the cornea measures a corneal stromal thickness of 300 microns or less in areas of the cornea, indicative of post-LASIK ectasia. The subject is chosen to receive treatment using short wavelength ultraviolet irradiation following administration of a low concentration photoinitiator. Drops of 0.35M sodium persulfate in 0.02M sodium phosphate buffer at a pH of 7.6-8.0 are administered to the cornea in an applicator designed to limit exposure to the location of keratoconus on the corneal surface. The cornea is then exposed to one 10-second burst of ultraviolet irradiation at an intensity of 1000 mW.

After treatment, the subject shows significant improvement in vision and a two-fold increase in CH, indicating an increase in the biomechanical integrity of the cornea.

### Example 4

### Corneal Stabilization Following Orthokeratology Lens Wear

Subjects are fitted with lenses for orthokeratology. The subjects have one (1) eye treated by exposure to ultraviolet irradiation following pretreatment with a photoinitiator. The contralateral eye is an untreated control. Selection of the eye to be treated is random. Examinations at the initial visit and at each follow-up include unaided visual acuity, slit-lamp examination, refractive error, corneal topography, and corneal hysteresis using the Reichert Ocular Response Analyzer. CH values correlate with reduced mechanical stability. The subject is chosen to receive treatment using short wavelength ultraviolet irradiation following administration of a low concentration photoinitiator. Drops of 0.35M sodium persulfate in 0.02M sodium phosphate buffer at a pH of 7.6-8.0 are administered to the cornea in an applicator designed to limit exposure to the corneal surface. The cornea is then exposed to one 10-second burst of ultraviolet irradiation at an intensity of 1000 mW.

Results show minimal to no regression of visual acuity in the treated eye compared to the untreated or control eye demonstrating effectiveness of ultraviolet irradiation to stabilize the cornea following vision correction using orthokeratology lens wear.

### OTHER EMBODIMENTS

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention herein. Such equivalents are intended to be encompassed in the scope of the present invention.

All references, including patents, publications, and patent applications, mentioned in this specification are herein incorporated by reference in the same extent as if each independent publication, patent or patent application was specifically and individually indicated to be incorporated by reference.

We claim:

1. A method of treating keratoconus, comprising applying a photoinitiator to the keratoconic cornea and exposing the cornea to ultraviolet irradiation for a period of equal to or less than about 10 minutes.

2. The method of claim 1, wherein the photoinitiator is chosen from sodium persulfate, potassium persulfate, ammonium persulfate, sodium thiosulfate, ferrous chloride tetrahydrate, or sodium bisulfate.

3. The method of claim 2, wherein the photoinitiator is sodium persulfate.

4. The method of claim 1, wherein the ultraviolet irradiation has a wave length in the range of about 254 nm to about 400 nm.

5. The method of claim 4, wherein the ultraviolet irradiation has a wave length of about 310 nm to about 400 nm.

6. The method of claim 1, wherein the intensity of the ultraviolet light is in the range of about 100 mW to about 1000 mW.

7. The method of claim 6, wherein the intensity of the ultraviolet light is about 1000 mW.

8. The method of claim 1, where in the period of exposure comprises from 1 to 4 bursts of ultraviolet light exposure, wherein each burst of ultraviolet exposure is about 10 seconds in duration and the period of non-ultraviolet exposure between each burst is about 10 seconds.

9. The method of claim 8, wherein the period of ultraviolet light exposure consists of a single exposure of about 10 seconds.

10. A method of treating ectasia following a corneal surgery, comprising applying a photoinitiator to the ectasic cornea and exposing the cornea to ultraviolet irradiation for a period of equal to or less than about 10 minutes.

11. The method of claim 10, wherein the photoinitiator is chosen from sodium persulfate, potassium persulfate, ammonium persulfate, sodium thiosulfate, ferrous chloride tetrahydrate, or sodium bisulfate.

12. The method of claim 11, wherein the photoinitiator is sodium persulfate.

13. The method of claim 10, wherein the ultraviolet irradiation has a wave length in the range of about 254 nm to about 400 nm.

14. The method of claim 13, wherein the ultraviolet irradiation has a wave length of about 310 nm to about 400 nm.

15. The method of claim 10, wherein the intensity of the ultraviolet light is in the range of about 100 mW to about 1000 mW.

16. The method of claim 15, wherein the intensity of the ultraviolet light is about 1000 mW.

17. The method of claim 10, where in the period of exposure comprises from 1 to 4 bursts of ultraviolet light exposure, wherein each burst of ultraviolet exposure is about 10 seconds in duration and the period of non-ultraviolet exposure between each burst is about 10 seconds.

18. The method of claim 17, wherein the period of ultraviolet light exposure consists of a single exposure of about 10 seconds.

19. The method of claim 10, wherein the surgery is chosen from Laser Assisted In Situ Keratomielusis (“LASIK”), Laser Epithelium Keratomileusis (“E-LASIK”), Conductive Keratoplasty (“Radiofrequency energy”), or Microwave Thermokeratoplasty.

20. The method of claim 19, wherein the surgery is LASIK.

21. A method of strengthening a cornea prior to a corneal surgery, comprising applying a photoinitiator to the cornea and exposing the cornea to ultraviolet irradiation for a period of equal to or less than about 10 minutes, followed by the corneal surgery.

22. The method of claim 21, wherein the photoinitiator is chosen from sodium persulfate, potassium persulfate, ammonium persulfate, sodium thiosulfate, ferrous chloride tetrahydrate, or sodium bisulfate.

23. The method of claim 22, wherein the photoinitiator is sodium persulfate.

24. The method of claim 21, wherein the ultraviolet irradiation has a wave length in the range of about 254 nm to about 400 nm.

25. The method of claim 24, wherein the ultraviolet irradiation has a wave length of about 310 nm to about 400 nm.

26. The method of claim 21, wherein the intensity of the ultraviolet light is in the range of about 100 mW to about 1000 mW.

27. The method of claim 26, wherein the intensity of the ultraviolet light is about 1000 mW.

28. The method of claim 21, where in the period of exposure comprises from 1 to 4 bursts of ultraviolet light exposure, wherein each burst of ultraviolet exposure is about 10 seconds in duration and the period of non-ultraviolet exposure between each burst is about 10 seconds.

29. The method of claim 28, wherein the period of ultraviolet light exposure consists of a single exposure of about 10 seconds.

30. The method of claim 21, wherein the surgery is chosen from Laser Assisted In Situ Keratomielusis (“LASIK”), Laser Epithelium Keratomileusis (“E-LASIK”), Conductive Keratoplasty (“Radiofrequency energy”), or Microwave Thermokeratoplasty.

31. The method of claim 30, wherein the surgery is LASIK.

32. The method of claim 21, wherein the ultraviolet irradiation treatment precedes the surgery by about one to two weeks.
